(19) United States
(12) Plant Patent Application Publication
(10) Pub. No.: US 2015/0106986 P1
(43) Pub. Date: Apr. 16, 2015

(54) Buddleia plant named 'PIIBD-II'

(50) ;

(71) Applicants: Michael A. Dirr, Bogart, GA (US); Joshua H. Kardos, Watkinsville, GA (US)

(72) Inventors: Michael A. Dirr, Bogart, GA (US); Joshua H. Kardos, Watkinsville, GA (US)

(73) Assignee: Plant Introductions, Inc., Watkinsville, GA (US)

(21) Appl. No.: 13/998,257

(22) Filed: Oct. 16, 2013

(51) Int. Cl. A01H 5/00

(52) U.S. Cl. PLT/242

(57) ABSTRACT

A new and distinct cultivar of Buddleia plant named ‘PIIBD-II’, characterized by its compact, rounded to upright growth habit, dark green foliage, reddish pink flowers with an orange center, and long flowering season, with flowers produced from summer to the first frost.

### GENUS AND SPECIES OF PLANT CLAIMED

Buddleia hybrid Franch.

### VARIETY DENOMINATION

‘PIIBD-II’

### BACKGROUND OF THE INVENTION

The present invention relates to a new and distinct cultivar of Buddleia plant, botanically known as Buddleia hybrid Franch., commonly known as butterfly-bush, and hereinafter referred to by the cultivar name ‘PIIBD-II’. ‘PIIBD-II’ is grown primarily as an ornamental for landscape use and for use as a potted plant.

‘PIIBD-II’ originated from an open-pollination of the cultivar ‘Miss Ruby’ (U.S. Plant Pat. No. 19,950) growing in Watkinsville, Ga. in 2010. The cultivar ‘PIIBD-II’ originated and was selected in a cultivated environment in Watkinsville, Ga. from the progeny of this open-pollination by continued evaluation for growth habit, foliage and flower characteristics.

Asexual reproduction of by stem cuttings in Watkinsville, Ga. since 2011 has shown that all the unique features of this new Buddleia, as herein described, are stable and reproduced true-to-type through successive generations of such asexual propagation.

### SUMMARY OF THE INVENTION

Plants of the new cultivar ‘PIIBD-II’ have not been observed under all possible environmental conditions. The phenotype may vary somewhat with changes in light, temperature, soil and rainfall without, however, any variance in genotype.

The following traits have been observed and represent the characteristics of the new cultivar. In combination these characteristics distinguish ‘PIIBD-II’ from all other varieties in commerce known to the inventor. 1. Compact, rounded to upright growth habit; 2. Dark green foliage; 3. Reddish pink flowers with an orange center; 4. Long flowering season, with flowers produced from summer to the first frost.

‘PIIBD-II’ is distinguished from its female parent, ‘Miss Ruby’, by its flower color and inflorescence size. ‘PIIBD-II’ has darker reddish pink flower color and larger inflorescences, whereas ‘Miss Ruby’ has lighter medium pink flower color and smaller inflorescences.

‘PIIBD-II’ can be compared to ‘Miss Molly’ (U.S. Plant Pat. No. 23,425), but differs in the following characteristics. has larger foliage, darker reddish pink flower color and larger inflorescences, whereas ‘Miss Molly’ has smaller foliage, lighter reddish purple flower color and smaller inflorescences.

### BRIEF DESCRIPTION OF THE DRAWINGS

The accompanying color photographs illustrate the flower and foliage characteristics and the overall appearance of ‘PIIBD-II’, showing the colors as true as it is reasonably possible to obtain in color reproductions of this type. Colors in the photographs may differ slightly from the color values cited in the detailed botanical description which accurately describe the colors of the new Buddleia.

FIG. 1 Illustrates the overall appearance of a plant of ‘PIIBD-II’ planted in the ground.

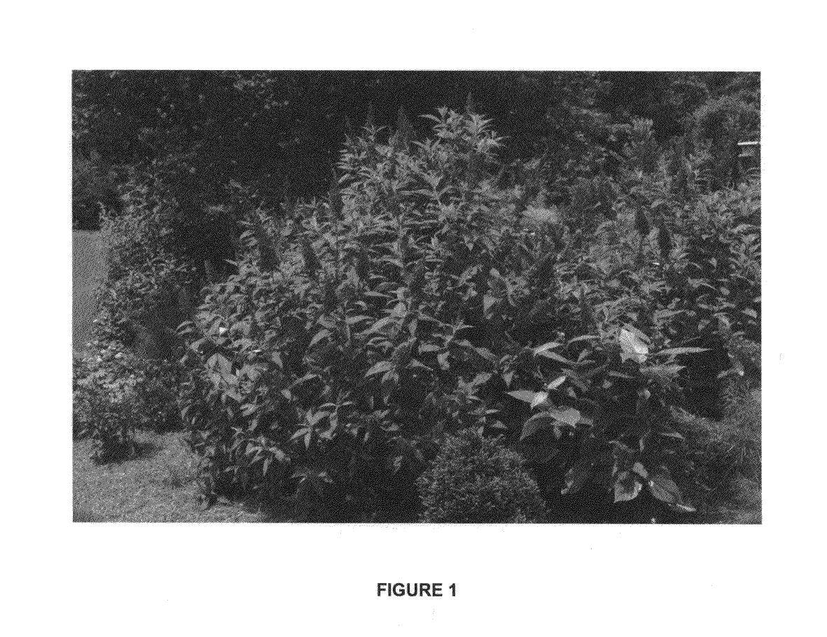

FIG. 2 Illustrates a close-up view of the inflorescences of ‘PIIBD-II’.

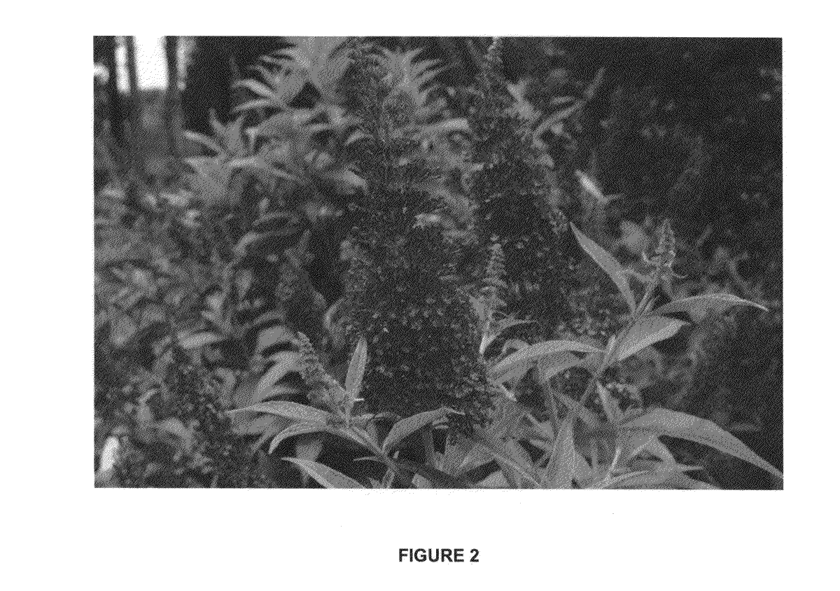

### DETAILED DESCRIPTION

In the following description, color references are made to The Royal Horticultural Society Colour Chart, 2007 Edition, except where general terms of ordinary dictionary significance are used. The plant used for the description was approximately three years old and was grown in the ground in a nursery in Watkinsville, Ga. Colors are described using The Royal Horticultural Society Colour Chart (R.H.S.).
Botanical classification: Buddleia hybrid Franch. ‘PIIBD-II’. Parentage: Female, or seed, parent: ‘Miss Ruby’ (U.S. Plant Pat. No. 19,950). Male, or pollen parent: unknown (open-pollinated). Propagation: terminal cuttings. Time to initiate roots, summer: about 21 days at 32° C.
Plant description: Deciduous flowering shrub; compact, rounded to upright growth habit. Freely branching; pruning enhances lateral branch development.
   Root description.—numerous, fibrous and well-branched.
   Plant size.—about 213 cm high from the soil level to the top of the inflorescences and about 234 cm wide. First year stems have a diameter of about 3.5 mm. Shape: quadrangular when young, becoming rounded with age. Pubescence: tomentulose when young, becoming glabrous with age. Second year and older stems have a diameter of about 4.5 mm or more. Shape: round.
   Trunk diameter.—3.8 cm at the soil line. Color: N199C.
   Internode length.—about 3.9 cm.
   Strength.—flexible when young, becoming stronger with age.
   First year stem color (young).—194B, at times overlaid with 178A. Color (mature): 199A.
   Second year and older stem color.—199A. Bark does not exfoliate.
Vegetative buds: Opposite arrangement, naked, subglobose, 2 bud scales with tomentulose pubescence. Color: 197D. Size: about 2 mm in length and 1 mm in width.
Foliage description:
   Arrangement.—opposite, simple. Leaf length: about 8.6 cm. Leaf width: about 2.5 cm. Shape: lanceolate to elliptical. Apex: acuminate. Base: cuneate. Margin: closely serrate to serrulate.
   Texture.—upper surface is glabrous, lower surface is tomentose.
   Venation pattern.—pinnate. Venation color (upper surface): 144A. Venation color (lower surface): 144C.
   Foliage color (upper surface).—N137A. Foliage color (lower surface): 194A.
   Petiole length.—about 3 mm. Petiole diameter: about 1.5 mm. Petiole color: 144C. Pubescence: tomentulose.
Flower description: Flowers on new growth. Flowers are produced from about June through the first frost in Watkinsville, Ga. An inflorescence is showy for about three weeks, and individual flowers last about three days and are self-cleaning. Approximately 265 flowers per individual inflorescence.
   Inflorescence type.—terminal panicle. Inflorescence length: about 16 cm. Inflorescence width: about 4.9 cm. Fragrance: pleasant and sweet.
   Peduncle.—about 14.6 cm in length, about 2 mm in diameter, color is 146C, at times overlaid with 178A, with tomentulose pubescence.
   Individual flowers.—about 1.2 cm in height and 8 mm in diameter and salverform in shape.
   Flower buds.—Length: about 8 mm; Diameter: about 2 mm; Color: 71B. Shape: elongated, linear balloon.
   Pedicels.—about 2.5 mm in length, less than 1 mm in diameter, 146C in color with tomentulose pubescence.
   Calyx.—consists of 4 sepals fused at the base. The calyx is about 3 mm in length, about 1 mm in diameter, 147B in color, tubular in shape with tomentulose pubescence. The individual sepals are lanceolate in shape, with an acute apex and entire margin, 147B in color with tomentulose pubescence.
Petals:
   Arrangement/appearance.—4 petals fused at the base to form the corolla tube, which is about 9 mm in length, 1 mm in width, outer surface is 71A in color and the inner surface or center of the flower is N172C in color.
   Petal.—Length: about 4 mm. Width: about 4 mm. Shape: broad spatulate. Apex: broad obtuse to rounded. Margin: ruffled. Texture: glaucous.
   Petal color.—upper and lower surfaces are 71A in color.
Stamens:
   Quantity/arrangement.—4 stamens per flower, fused to the inner base of the corolla tube, about 4 mm long. Filaments: 3 mm in length, less than 0.5 mm in width, and 1D in color. Anthers: 1 mm in length, 0.5 mm in width, and 4C in color. The stamens are not pubescent. Pollen: produced in small quantities and is 158D in color.
Pistils:
   Quantity.—One superior pistil per flower. Pubescence: none. Pistil: about 5 mm in length and about 1 mm in width. Stigma: elongated shape, about 1.5 mm in length and 0.5 mm in width and 144C in color. Style: tubular in shape, about 1 mm in length, less than 0.5 mm in width and 73D in color. Ovary: ovoid in shape, about 2.5 mm in length, about 1 mm in width and 144B in color.
Fruit:
   Type/appearance.—two-valved, septicidally dehiscent capsule. Length: about 7 mm. Width: about 2 mm. Immature color: 144C. Mature color: N200D. Each capsule contains many tiny seeds 165D in color.
Disease/pest resistance: ‘PIIBD-II’ has not exhibited any particular pest or disease susceptibility or resistance, except occasional susceptibility to spider mites under very hot and dry conditions. ‘PIIBD-II’ is cold hardy to USDA Hardiness Zones 5-9.

I claim:

1. A new and distinct Buddleia plant named ‘PIIBD-II’, as illustrated and described herein.
